(19) United States
(12) Patent Application Publication
(10) Pub. No.: US 2005/0255160 A1
(43) Pub. Date: Nov. 17, 2005

(54) Polymide resin dermal composition

(76) Inventor: Stephen Bell, Harwinton, CT (US)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/843,236

(22) Filed: May 11, 2004

(51) Int. Cl. A61K 9/14

(52) U.S. Cl. 424/486

(57) ABSTRACT

A gel composition made from a polyamide resin combined with an lower carboxylic acid or anhydride alkyl amide; the compositions are suitable for making attractive topically-applied cosmetic, topical fragrance and topical dermatology drug products in translucent gel form.

### RELATED APPLICATIONS

None

### GOVERNMENT INTEREST

None

### FIELD OF THE INVENTION

My invention relates to topical carriers. My invention is suitable for making clear or pigmented cosmetic, fragrance and active ingredient (e.g., dermatology pharmaceutical or “active cosmetic” ingredient) topical products. These products can be for the protection of, the treatment of, enhancing the appearance of, or the physical preservation or improvement of, the skin (or its “appendages,” the fingernails, the hair, et cetera). My invention is a composition made from a polyamide resin combined with an alkyl amide of a mono-, di- or tri-carboxylic acid.

My invention can be used to make clear and pigmented gels, gel solids, anhydrous silicone/oil emulsions, and other cosmetic forms which can be applied to skin. In particular, my invention can be used to make improved cosmetic, drug, fragrance and dermatology products in stick and gel form, with enhanced feel, color deposition and gloss. These inventions may be used alone or as a part of an emulsion system whereby the gel system is segregated from the body of the emulsion; segregation may improve the stability, efficacy, or cosmetic application or “feel” properties.

### BACKGROUND

Cosmetic sticks, pencils, and gels are common vehicles for the deposition of actives in general, and color in particular, to the face and body for treatment and or decorative purposes. Solid cosmetic compositions used to deliver actives and color in particular are usually made of waxes such as beeswax, candellila, carnauba, triglycerides in combination with oils and pigments or acid soaps.

Sticks or other solid forms are created commercially by melting waxes and soaps, adding liquid oils and pigments to the melted wax or soap, and then thoroughly mixing, after which the molten composition is cast in a mould. After cooling, the wax or soap provides a solid structure to the liquid oil and pigment. In the case of lipsticks, for example, the solid stick is placed in a lipstick case. Pencils, however, can be molded, then placed in slotted wood, which are subsequently glued together, or the molten material can be directly molded into a pre-formed shell or retractable package. In yet another case, the molten material can be poured into metal pans or plastic “pots.”

An important disadvantage to the current art of making solid anhydrous compositions is the lack of smoothness on application, color deposition and gloss. These disadvantages are inherent in the use of traditional waxes, which are opaque and crystalline. The crystalline nature of wax is an important impediment to making a solid anhydrous product, because wax can lack smoothness on application, generally has a poor, uneven or inadequate deposition of the color pigment or the active ingredient, and exhibit poor gloss. Another shortcoming is the high degree of shrinkage in the cast product, when the wax cools from the high molten casting temperature to the low crystallization temperature.

In an attempt to overcome these disadvantages, researchers have suggested the use of structurants and gellants that are non-crystalline in nature. One such proposed route involves the use of polyamide resins and its esters. [WHERE WAS THIS SUGGESTED? PLEASE FILL IN CITATION TO THE SOURCE OF THIS ASSERTION] The problem with polyamide resins and its esters, is that polyamide resin structures that have a fragile enough physical matrix to deposit on the skin when rubbed, will generally have a physical matrix fragile enough to result in syneresis—pooling of oil on the surface of the molded structure. In order to avoid syneresis, the molded structure has to be significantly harder, with a stronger physical matrix; such stronger physical matrices are usually too strong to erode when rubbed on the skin, and thus fail to deposit pigment and or the active ingredient on the skin.

Another attempt, disclosed in U.S. Pat. No. 6,423,324, teaches the use of dimer polyamides (Versamid™ polyamide and Uni-Rez™ polyamide) with N-Acyl amino acid amide as the gelator.

Another attempt [is this the '324 Cosmolabs patent discussed above? If not, where is this attempt documented/published?] involves the use of n-acyl amino acid gelling agents, in combination with waxes, to form a structure for antiperspirants.

I have found a way to make an improved cosmetic composition with improved gloss and deposition of pigments onto the skin. My invention originates as a translucent gel composition—thus maximizing the gloss of the applied finish and, in the application of color cosmetics, also maximizes the depth and intensity of the applied color.

My invention may be made into an improved gel-solid stick, or another cast or molded form, to enhance the gloss and pigment deposition of an already-known cosmetic product. Further, my invention can be used to make a stable gel-stick, gel-solid structure that deposits pigments and actives to the skin, while avoiding the adverse effect of cosmetically-unacceptable syneresis.

### SUMMARY

My invention is a combination of a polyamide gallant and an alkyl amide gallator, combined to make a gel. The gel can be made as a translucent (colorless or pigmented) or opaque gel, and may be anhydrous or solid. It is shelf-life stable, and it does not exhibit cosmetically-unacceptable synerisis. It is made by combining a polyamide resin with an alkyl amide.

### DETAILED DESCRIPTION

The Polyamide Resin

The polyamide resin is a monomer resin. It may be modified to have alkyl, silicone and/or fluorocarbon terminal end groups. Polyamide resin is commonly referred to as nylon; it is the reaction product of an alkyl di-carboxylic acid of the formula:
HOOC—R—COOH,
where R is an alkyl group with chain length of C₁-C₁₀₀ and an alkyl diamine of the formula:
H₂N—R—NH₂
where R is an alkyl group with chain length of C₁-C₁₀₀ to give a polymer of the formula:
HOOC[(CH2)ₐCONH(CH2)_{b}NHCO(CH₂)_{c}]ₓCOOH
Wherein a, b, c are integers from 1-100. Examples of polyamide resins include, but are not limited to, the Macromelt™ resins, commercially available from Henkel Corporation ______, and the Uniclear™ and Sylvaclear™ resins, commercially available from Arizona Chemical Corporation, ______. Polyamide resins may be used singly or in combination with another polyamide resin(s).

The polyamide resin(s) may also have functional end groups, such as ester or amide groups.

The Alkyl Amide

The alkyl amide can be a mono-, di- or tri-basic carboxylic acid or -carboxylic anhydride, alone or in combination. The alkyl group can have a carbon number anywhere from about 6 to about 30. The carbon number of the mono-, di- or tri-basic carboxylic acid or carboxylic anhydride can be from 1 up to about 30. I currently prefer as gelling agents, N-lauroyl-glutamic acid diethyl amide, N-lauroyl-glutamic acid dihexyl amide, N-lauroyl-glutamic acid dibutyl amide, N-lauroyl-glutamic acid dihexyl amide, N-lauroyl-glutamic acid dioctyl amide, N-lauroyl-glutamic acid didecyl amide, N-lauroyl-glutamic acid didodecyl amide, N-lauroyl-glutamic acid distearyl amide, N-lauroyl-glutamic acid diheptyl amide, N-stearoyl-glutamic acid dibutyl amide, N-staroyl-glutamic acid diethyl amide, N-stearoyl-glutamic acid dioctyl amide, and blends thereof. These are not the only acceptable gelling agents, however.

Acceptable gelling agents include any alkyl amide of a mono-carboxylic acid, of a di-carboxylic acid, and of a tri-carboxylic acid. For simplicity, I refer to mono-, di- and tri-carboxylic compounds as “lower carboxylic” compounds. Thus, “lower carboxylic acid” means a mono-, or a di- or a tri-carboxylic acid.

Similarly, also acceptable are alkyl amides of carboxylic anhydrides. I thus use the short-hand “carboxylic compound” to designate carboxylic acid and carboxylic anhydride. Thus, “lower carboxylic compound” means any mono- or a di- or a tri-carboxylic acid, and any mono- or a di- or a tri-carboxylic anhydride.

Other Ingredients

The polyamide resin—alkyl amide combination can also include an anhydrous liquid carrier, such as a polar or non-polar oil, an ester, a paraffin, an isoparaffin, an alcohol, a silicone, an alkyl silicone, a cyclic silicone, or a silicone ether.

To make a finished cosmetic product, one can also add colorants or pigments, fragrance, flavor, or an active ingredient, or herbal, plant, or animal extracts. These may be mixed as-is, or provided in encapsulated, bound, or adsorbed form, et cetera.

Colorants and pigments are known in the art, and include, for example: cosmetically-accepted pigments of metal oxides and hydroxides, organic pigments as dyes and/or lakes such as, but not limited to, D&C Red Nos.: 6, 7, 21, 27, 30, 33 or 40; yellow Nos. 5, 6 or 10, or Blue No. 1. Pigments generally accepted for use in cosmetic products include, for example: iron oxides, titanium dioxide, zinc oxide, D&C Colors such as Red No. 6 Barium lake, Red No. 7 Calcium lake, Red No. 21 Aluminum lake, Red No. 27 aluminum lake, Red No. 33 Aluminum lake, Red No. 49 Aluminum lake, Yellow No. 5 Aluminum lake, yellow No. 6 Aluminum lake, Blue No. 1 Aluminum lake, and (approved for use in the European Community, but not yet approved in the United States of America) Carbon black. The pigment may be provided as-is, or surface-modified, with the surface modified chemically or physically using silicones, such as dimethicone, alkyl akoxy silanes, methicone, dimethicone crosspolymers, fluorosilicones, soaps, acid soaps, fatty acids, synthetic surfactants, phosphated fluorosilicones and lecithin.

One may also add cosmetically- or pharmaceutically-active substances such as anti-inflammatory, analgesics, enzymes, retinoids, anti-infective, antiperspirant, vitamins, exfoliants, anti-infectives, antiperspirants, anti-pruritics, sunscreens, and other compounds that have some benefit when applied topically or trans-dermally.

One can also add fillers. These are known in the art and include, for example, pearlescent materials like mica or talc, barium sulphate, calcium carbonate, clay, waxes, acid soaps, soaps, carbide, silicas, graphite and particulate nylon. Fillers such as titanium-coated mica, metal pearls such as copper and aluminum, mica, talc, starch, particulate nylon, clays, soaps, acid soaps, fatty acids, waxes, carbide, graphite, oil soluble polymers, polymethyl methacrylate, barium sulphate, calcium carbonate, titanium coated mica, bentonite, hectorite, AL/Mg hydroxide state, trihydroxystearate. Pigments and fillers can be used as-is, or surface-modified, where the surface is modified chemically or physically using, for example, silicones such as dimethicone, alkyl alkoxy silanes, methicone, dimethicone crosspolymers, fluorosilicones, phosphated fluorosilicones or lecithin. Other surface modifications are known in the art

Other fillers include anhydrous liquid carriers such as polar and non polar oils, esters, paraffin, isoparaffin, alcohols, silicones, alkyl silicones, cyclic silicones or silicone ethers. For example, the filler(s) may be an anhydrous oil, such as saturated, unsaturated, branched and cyclic hydrocarbons of carbon length C₁-C₆₀; saturated, unsaturated, branched and cyclic alcohols of carbon length C₁-C₆₀; saturated, unsaturated and branched: mono, di, tri-carboxylic including dimer and trimer acids of carbon length of C₂-C₆₀; silicones, alkyl silicones, cyclic silicones, silicone ethers such as DC556™, DC200™, DC5200™, DC245™ or DC345™, each commercially available from Dow Corning Corporation, Corning, N.Y.; saturated, unsaturated triglycerides, diglycerides; and polyurethanes, polyurethane copolymers and prepolymers.

The gel may optionally include other co-gellant or polymeric materials, such as natural cellulosic polymers, including guar, ethyl and carboxymethyl cellulose, linear polyethylene, and modified polyethylene such as Enhance AG-50/200™, hectorite clays, and polyurethane.

The polyamide-alkyl amide gel mixture may be used as is, or as a part of an emulsion system whereby the polyamide-alkyl amide gel system is segregated from the body of the emulsion for purposes of stability, efficacy or cosmetic application or feel properties.

Given the basic composition of the gel, and the list of potential ingredients, I now turn to discuss some examples of compositions made using my invention.

### EXAMPLES

The following examples are not limited to the range of formulation but illustrate specific embodiments of various facial cosmetic compositions. For thoroughness, I discuss a method of manufacture. In the ratios provided in the Examples below, the polyamide resin acts as a gellant, and the alkyl amide as a gellator.

Each composition exemplified here is prepared by mixing the components and heating them to melting (about 100° C., depending on the specific components). The components may be heated to melting either before mixing them all together, so one can mix liquids, or heat them only after mixture, thus requiring mixture of a semi-solids and liquids. Once melted and mixed, the mix is maintained molten and agitated until a clear homogeneous liquid is achieved. I prefer to add the polyamide resin after the other components are molten and mixed; the resulting mixture is then agitated until the polyamide resin completely dissolves.

Any pigments are ground with one of the oils in the formulation, to disperse the pigment. This may be done using conventional grinding equipment, such as a three-roller mill, a ball mill, or a media mill. I prefer the pigment be ground to achieve a particle size of 10 microns or less, to assure adequate dispersion. Once the pigment is ground to the desired particle size, it is added to the rest of the molten formulation (molten is perhaps 80 degrees centigrade or above). The heated liquid—pigment blend is then poured into a lipstick mould, a pot, a pan, or a pencil form.

Table 1 recites various examples of stick formulations incorporating an alkyl amide gelling agent and a polyamide resin. [PLEASE CHECK THE FOOTNOTES; THERE WAS ONE FOOTNOTE MISSING IN THE DRAFT I RECEIVED]

TABLE 1

| Component | Example 1 | Example 2 | Example 3 |
|---|---|---|---|
| Octyl dodecanol | 49.0 | 46.0 | 44.0 |
| Polyisobutene¹ | 5.0 | 6.0 | 10.0 |
| Dibutyl lauroyl glutamide² | 5.0 | 5.0 | 5.0 |
| Dimer Acid³ | 6.0 | 6.0 | 6.0 |
| PVP/Hexadecene⁴ | 7.0 | 9.0 | 8.0 |
| Polyglyceryl-3 diisostearate⁵ | 4.0 | 5.0 | 6.0 |
| Lauryl dimethicone copolyol⁶ | 8.0 | — | 4.0 |
| Polyamide resin⁷ | 8.0 | 7.0 | 6.0 |
| Wrapped titanium dioxide⁸ | 3.0 | 5.0 | 6.0 |
| Wrapped D & C Red No. 7 | 2.0 | 3.0 | 5.0 |
| Ca. Lake¹⁰ | | | |
| Wrapped Timica Nu-antique | 3.0 | 8.0 | — |
| gold¹ | | | |

¹Henkel Eutanol G ™; Jarchem Jarcol I-20 ™
²Lipo Panalane ™ 300
³Ajinomoto GP-1 ™
⁴Henkel Empol ™ 1062
⁵ISP Ganex ™ V216
⁶Dow Corning DC5200 ™
⁷Henkel Macromelt ™ 6040
⁸Trend Cosmetics Inc. Chromawrap ™ Titanium Dioxide
⁹Trend Cosmetics Inc. Chromawrap ™ Red No. 7 Calcium Lake
¹⁰Trend Cosmetics Inc. Antique gold ™

Based on the foregoing discussion, one of skill in the art can easily develop variations of my examples. Thus, I intend my patent to cover not just the specific Examples I list here, but the material defined by the legal claims appended here, and their legal equivalents.

In the claims, I use the term “a” to mean one or more. That is to say, for example, “a lower carboxylic acid” encompasses one lower carboxylic acid alone, as well as several lower carboxylic acids combined.

1. A composition of matter comprising: a gel made from reacting polyamide resin with a lower carboxylic compound to produce said gel.

2. The composition of matter of claim 1, said gel comprising from about 0.1% to about 40% by weight of said polyamide resin, and from about 0.1% to about 30% by weight of said lower carboxylic compound.

3. The composition of matter of claim 1, wherein said lower carboxylic compound includes an alkyl group with a carbon number of from about 6 to about 30.

4. The composition of matter of claim 1, wherein said lower carboxylic compound comprises lower carboxylic acid.

5. The composition of matter of claim 5, wherein the carbon number of said lower carboxylic acid is from 1 to about 30.

6. The composition of matter of claim 1, wherein said lower carboxylic compound is selected from the group consisting of: an alkyl amide of citric acid; glutamic acid; tricarballylic acid; aconitic acid; an alkyl amide of alkyl succinic acid; alkyl glutamic acid; an alkyl amide of citric anhydride; glutamic anhydride; tricarballylic anhydride; aconitic anhydride; an alkyl amide of alkyl succinic anhydride; and alkyl glutamic anhydride.

7. The composition of matter of claim 1, wherein said lower carboxylic compound is selected from the group consisting of N-lauroyl-glutamic acid diethyl amide; N-lauroyl-glutamic acid dihexyl amide; N-lauroyl-glutamic acid dibutyl amide; N-lauroyl-glutamic acid dihexyl amide; N-lauroyl-glutamic acid dioctyl amide; N-lauroyl-glutamic acid didecyl amide; N-lauroyl-glutamic acid didodecyl amide; N-lauroyl-glutamic acid distearyl amide; N-lauroyl-glutamic acid diheptyl amide; N-stearoyl-glutamic acid dibutyl amide; N-staroyl-glutamic acid diethyl amide; and N-stearoyl-glutamic acid dioctyl amide.

8. The composition of matter of claim 2, further comprising a liquid carrier selected from the group consisting of: a polar oil; a non-polar oil; an ester; paraffin; isoparaffin; alcohol; polyurethane; silicone; alkyl silicone; cyclic silicone; and silicone ether.

9. The composition of matter of claim 8, said liquid carrier selected from the group consisting of a hydrocarbon of carbon length C₁-C₆₀; an alcohol of carbon length C₁-C₆₀; a silicone.

10. The composition of matter of claim 1, further comprising a cosmetically-acceptable pigment.

11. The composition of matter of claim 1, further comprising an active ingredient selected from the group consisting of: a flavorant; a plant derivative; an animal derivative; a drug substance; a vitamin; an exfoliant; an anti-infective, an anti-perspirant; an anti-pruritic; and a sunscreen.

12. The composition of matter of claim 1, further comprising an discontinuous phase, said discontinuous phase dispersed in said composition of matter of claim 1 to make an emulsion.

13. The composition of matter of claim 1, further comprising a continuous phase, said composition of matter of claim 1 dispersed in said continuous phase to make an emulsion.
